(19) United States
(12) Patent Application Publication
(10) Pub. No.: US 2015/0051599 A1
(43) Pub. Date: Feb. 19, 2015

(54) LIMITED-USE MEDICAL DEVICE

(71) Applicant: COVIDIEN LP, Mansfield, MA (US)

(72) Inventor: DANIEL A. JOSEPH, GOLDEN, CO (US)

(73) Assignee: COVIDIEN LP, Mansfield, MA (US)

(21) Appl. No.: 14/179,770

(22) Filed: Feb. 13, 2014

Related U.S. Application Data
(60) Provisional application No. 61/866,837, filed on Aug. 16, 2013.

(51) Int. Cl. A61B 18/14

(52) U.S. Cl. CPC A61B 18/1442
USPC 606/41

(57) ABSTRACT

A forceps comprises a reusable portion and a disposable portion configured to operably engage the reusable portion, the disposable portion including at least one limited-use feature, wherein upon disengagement of the disposable portion from the reusable portion, the at least one limited-use feature is transitioned from a first state, permitting engagement of the disposable portion with the reusable portion, to a second state, wherein the limited-use feature inhibits reengagement of the disposable portion with the reusable portion.

### CROSS REFERENCE TO RELATED APPLICATION

The present application claims the benefit of and priority to U.S. Provisional Application Ser. No. 61/866,837, filed on Aug. 16, 2013, the entire contents of which are incorporated herein by reference.

### BACKGROUND

1. Technical Field

The present disclosure relates to medical devices. More particularly, the present disclosure is directed to limited-use medical devices and medical devices including limited-use features.

2. Background of the Related Art

Certain medical devices (or components thereof) are capable of being used multiple times, and are thus referred to as reusable devices (or reusable components), while other medical devices (or components thereof) are configured for single use, and are thus referred to as disposable devices (or disposable components). Many such reusable and disposable medical devices, and/or the components thereof, are designed for a pre-determined number of uses and/or for a pre-determined usage time. Use of these devices beyond their prescribed usage time or number of uses may result in failure of the device, damage to the device, and/or injury to the patient or clinician. On the other hand, given the rising costs of performing medical procedures, clinician's have an incentive to maximize the reuse of medical devices (or components thereof).

### SUMMARY

Like reference numerals may refer to similar or identical elements throughout the description of the figures. As shown in the drawings and described throughout the following description, as is traditional when referring to relative positioning on a surgical instrument, the term “proximal” refers to the end of the apparatus that is closer to the user and the term “distal” refers to the end of the apparatus that is farther away from the user. The term “clinician” refers to any medical professional (e.g., doctor, surgeon, nurse, or the like) performing a medical procedure. The term “reusable” means that a device is capable of being used more than once. The term “sterilizable” means that a device is capable of being cleaned using a suitable procedure. To the extent consistent, any of the aspects and features described herein may be used in conjunction with any or all of the other aspects and features described herein.

In at least one aspect of the present disclosure, a forceps includes a reusable portion and a disposable portion configured to operably engage the reusable portion, the disposable portion including at least one limited-use feature, wherein upon disengagement of the disposable portion from the reusable portion, the at least one limited-use feature is transitioned from a first state, permitting engagement of the disposable portion with the reusable portion, to a second state, wherein the limited-use feature inhibits reengagement of the disposable portion with the reusable portion.

In another aspect of the present disclosure, the disposable portion is a disposable electrode assembly.

In another aspect of the present disclosure, the disposable electrode assembly is configured to conduct electrosurgical energy to tissue.

In another aspect of the present disclosure, the reusable portion is a jaw member disposed at a distal end of the forceps, the disposable portion is a jaw substrate that is operably engagable with the jaw member, and wherein the limited-use feature is a bifurcated anchor member disposed on the jaw substrate, the bifurcated anchor member being disposed in the first state before insertion into the jaw member, maintained in the first state after insertion into the jaw member, and transitioned to the second state upon disengagement from the jaw member.

In another aspect of the present disclosure, the bifurcated anchor member is made from a material having a low material failure such that removal of the bifurcated anchor member breaks at least a portion of the bifurcated anchor member, thus rendering the bifurcated anchor member inoperable for reinsertion into the jaw member.

In another aspect of the present disclosure, the reusable portion is a shaft member of the forceps and the disposable portion is a housing, and wherein the limited-use feature is a frangible anchor member that facilitates attachment of the housing to the shaft member.

In another aspect of the present disclosure, transition of the limited-use feature from the first state to the second state renders the disposable portion electrically inoperable.

In another aspect of the present disclosure, the disposable portion is configured to be disengaged from the reusable portion via a release member, wherein actuation of the release member transitions the limited-use feature from the first state to the second state.

In another aspect of the present disclosure, the limited-use feature further includes at least one mechanical fuse, and wherein, in the second state, the mechanical fuse prevents electrosurgical energy from being applied to tissue.

In another aspect of the present disclosure, the at least one mechanical fuse is configured to disconnect or break upon removal of the disposable portion from the reusable portion.

In another aspect of the present disclosure, a method for preventing reuse of a surgical instrument includes the steps of providing a forceps including a reusable portion and a disposable portion configured to operably engage the reusable portion, the disposable portion including at least one limited-use feature, wherein upon disengagement of the disposable portion from the reusable portion, the at least one limited-use feature is transitioned from a first state, permitting engagement of the disposable portion with the reusable portion, to a second state, wherein the limited-use feature inhibits reengagement of the disposable portion with the reusable portion, and disengaging the disposable portion from the reusable portion, thereby transitioning the limited-use feature from the first state to the second state.

In another aspect of the present disclosure, a forceps includes a reusable portion and a disposable portion configured to operably engage the reusable portion, the disposable portion including at least one limited-use feature, wherein upon disengagement of the disposable portion from the reusable portion, the at least one limited-use feature is transitioned from a first state, permitting engagement of the disposable portion with the reusable portion, to a second state, wherein the limited-use feature inhibits reengagement of the disposable portion with the reusable portion, wherein the limited-use feature includes a flexible clip configured to anchor the disposable portion to the reusable portion.

In another aspect of the present disclosure, the reusable portion includes a channel for accepting the flexible clip to allow the disposable portion and the reusable portion to snap-fit into engagement.

In another aspect of the present disclosure, the flexible clip is prevented from being removed from the channel without fracturing the flexible clip by applying a suitable force to fracture the flexible clip.

In another aspect of the present disclosure, the flexible clip is made of a brittle, crumplable, stretchable, or deformable material that allows disengagement under a desired force, but permanently disables flexible clip from reuse.

### BRIEF DESCRIPTION OF THE DRAWINGS

The above and other aspects, features, and advantages of the present disclosure will become more apparent in light of the following detailed description when taken in conjunction with the accompanying drawings, wherein:

FIG. 1 is a perspective view of a forceps provided in accordance with the present disclosure including a mechanical forceps, a disposable housing, and a disposable electrode assembly;

FIG. 2 is an enlarged, perspective view of a distal end of the forceps of FIG. 1;

FIG. 3 is a perspective view of the forceps of FIG. 1 with parts separated;

FIG. 4 is an enlarged, internal, side view of the disposable housing and the disposable electrode assembly of the forceps of FIG. 1, with parts partially removed;

FIG. 5 is a greatly-enlarged, perspective view of the disposable electrode assembly of the forceps of FIG. 1;

FIG. 6 is a greatly-enlarged, perspective view of one of the electrodes of the disposable electrode assembly of FIG. 1 with parts separated;

FIG. 7 is a greatly-enlarged, perspective view of the other electrode of the disposable electrode assembly of FIG. 1 with parts separated;

FIG. 8 is a side, perspective view of the forceps of FIG. 1 grasping tissue;

FIG. 9A is an enlarged, internal, perspective view of another embodiment of a disposable housing and disposable electrode assembly provided in accordance with the present disclosure, with parts partially removed, shown engaged with a forceps;

FIG. 9B is an enlarged, internal, perspective view of the disposable housing and disposable electrode assembly of FIG. 9, shown after disengagement from a forceps;

FIG. 10A is an enlarged, cross-sectional view of another electrode assembly, shown separated from a jaw member;

FIG. 10B is an enlarged, cross-sectional view of the electrode assembly of FIG. 10A, shown engaged to the jaw member;

FIG. 10C is an enlarged, cross-sectional view of the electrode assembly of FIG. 10A, shown after disengagement from the jaw member;

FIG. 11A is an exploded, perspective view of a release mechanism provided in accordance with the present disclosure and configured for use with a forceps similar to the forceps of FIG. 1;

FIG. 11B is an enlarged, top, cross-sectional view of the release mechanism of FIG. 11A, shown disengaged from the forceps;

FIG. 12A is a top, perspective view of a housing half provided in accordance with the present disclosure and configured for use with a forceps similar to the forceps of FIG. 1, the housing half including an anchor member; and

FIGS. 12B-12D are side, cross-sectional views of the housing half and anchor member of FIG. 12A, illustrating attachment and detachment of the housing half to the forceps.

### DETAILED DESCRIPTION

Particular embodiments of the present disclosure are described hereinbelow with reference to the accompanying drawings; however, the disclosed embodiments are merely examples of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

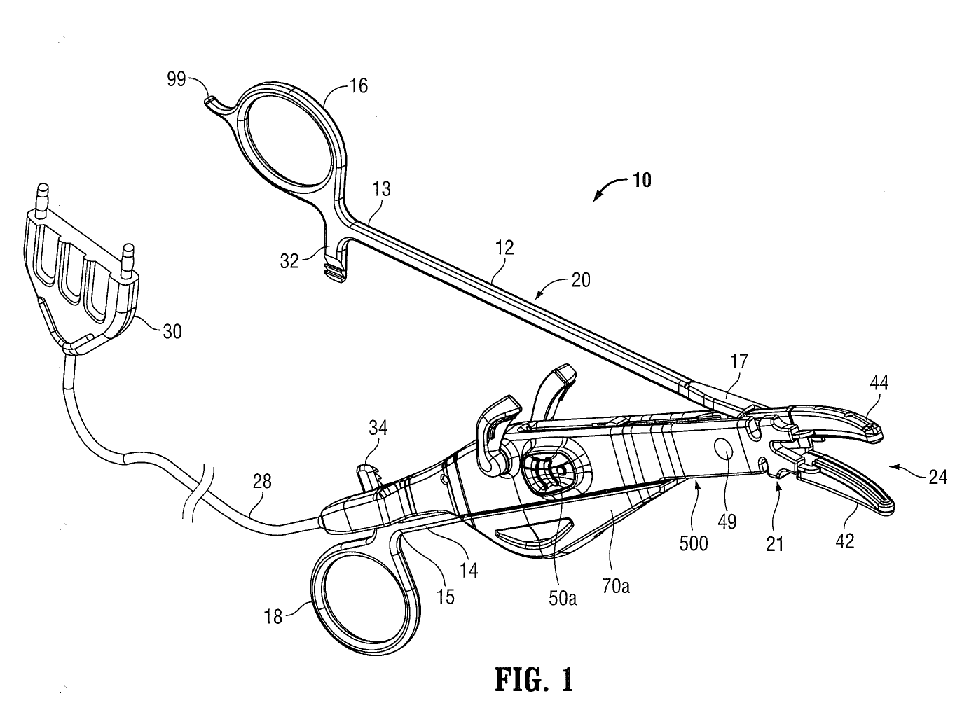

Referring now to FIG. 1, a forceps 10 for use in connection with open surgical procedures is shown, although forceps 10 may also be configured for use in connection with endoscopic surgical procedures. Alternatively, the present disclosure may be embodied in any other suitable medical devices such as, but not limited to scissors, staplers, probes, syringes, and any other electrical, mechanical, or electromechanical medical devices.

Disclosed herein are embodiments of a medical device having one or more removable components including at least one limited-use feature configured to limited use or reuse of the forceps. The present disclosure is described hereinbelow, by way of example only, with respect to a bipolar forceps 10 including a reusable portion 20 having an end effector 24 disposed at a distal end thereof and a disposable portion 500 including a disposable housing 70 and a disposable electrode assembly 21.

Continuing with reference to FIG. 1, reusable portion 20 includes first and second elongated shaft members 12 and 14. Elongated shaft member 12 includes proximal and distal end portions 13 and 17, respectively, and elongated shaft member 14 includes proximal and distal end portions 15 and 19, respectively. Disposed at proximal end portions 13, 15 of shaft members 12, 14 are handle members 16 and 18, respectively, that are configured to allow a user to effect movement of at least one of the shaft members 12 and 14 relative to the other. The end effector 24 includes opposing jaw members 42, 44 that extend from the distal end portions 17 and 19 of shaft members 12 and 14, respectively. The jaw members 42, 44 are movable relative to each other in response to movement of shaft members 12, 14.

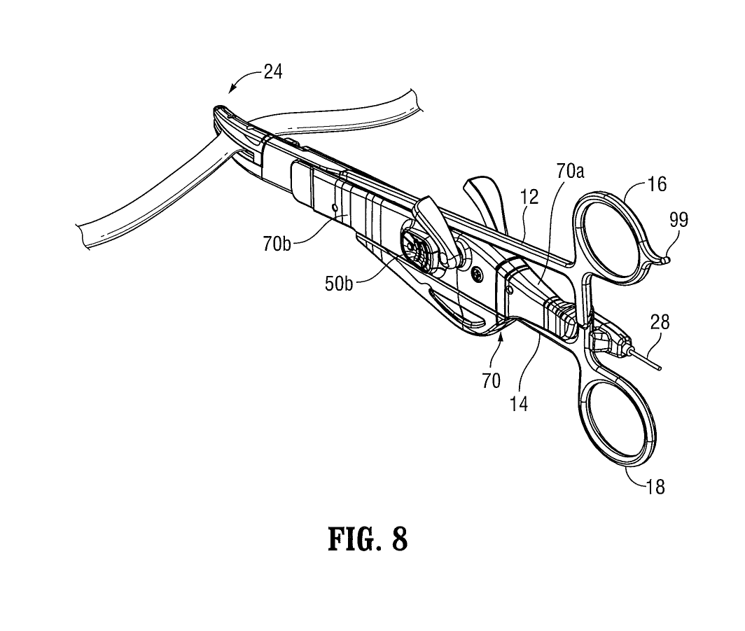

Shaft members 12 and 14 are affixed to one another about a pivot 25 such that movement of shaft members 12, 14, imparts movement of the jaw members 42, 44 from an open configuration (FIG. 1) wherein the jaw members 44, 42 are disposed in spaced relation relative to one another to a clamping or closed configuration (FIG. 8) wherein the jaw members 42, 44 cooperate to grasp tissue 150 therebetween.

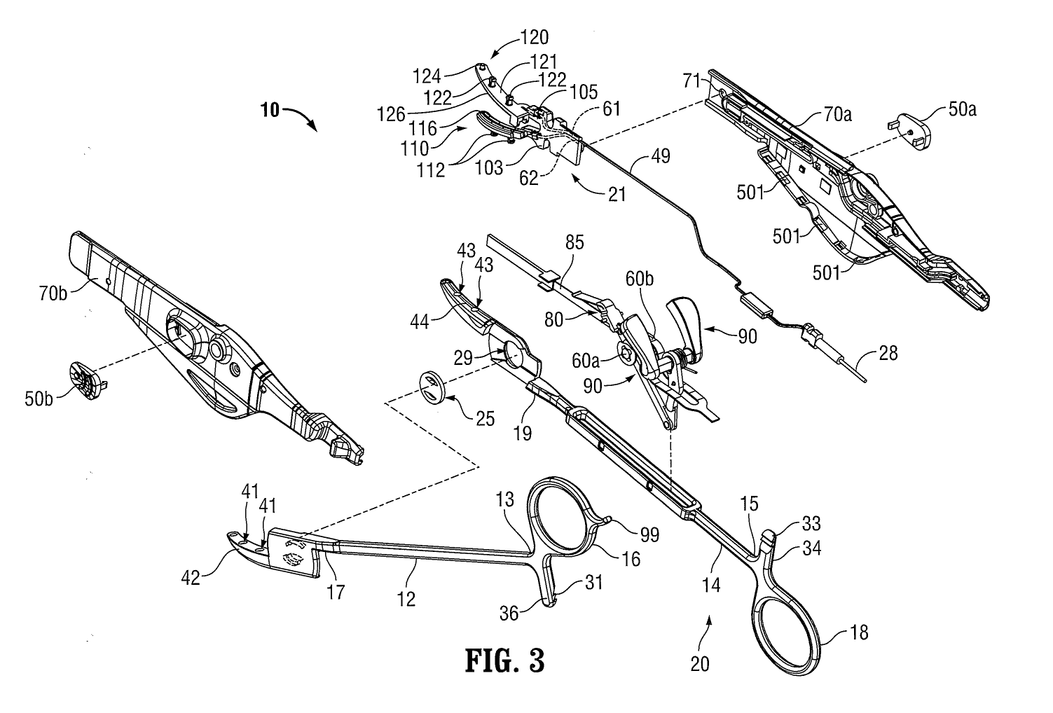

Each shaft member 12 and 14 also includes a ratchet portion 32 and 34 that extends from the proximal end portion 13, 15 of the respective shaft member 12, 14 towards the other ratchet in a generally vertically aligned manner such that the inner facing surfaces of each ratchet 32 and 34 abut one another when the shaft members 12, 14 are approximated. Each ratchet 32 and 34 includes a plurality of flanges 31 and 33 (FIG. 3), respectively, that project from the inner facing surface of each ratchet 32 and 34 such that the ratchets 32 and 34 may interlock at one or more positions. In some embodiments, each ratchet position holds a particular strain energy in the shaft members 12 and 14 to impart a specific closure force to the end effector 24. At least one of the shaft members, e.g., shaft member 12, includes a tang 99 that facilitates manipulation of forceps 10 during surgical conditions as well as facilitates attachment of electrode assembly 21 on reusable portion 20 as will be described in greater detail below.

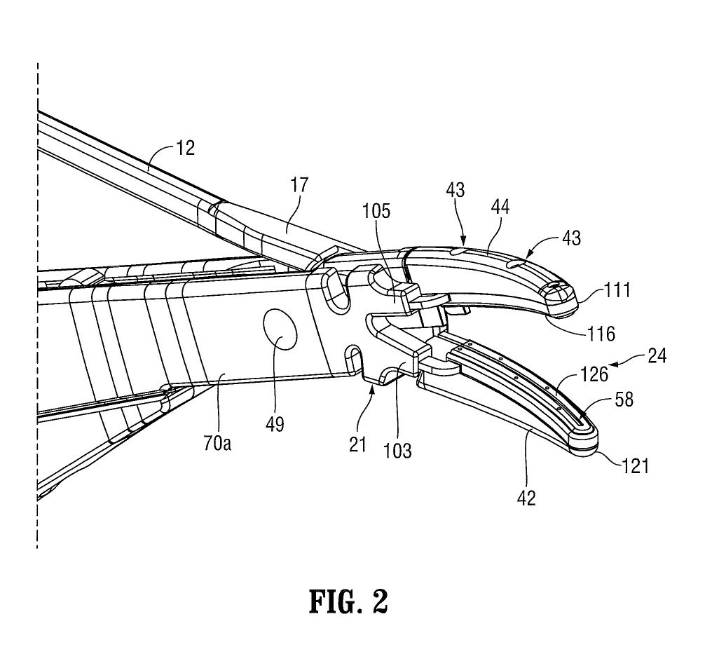

Referring to FIGS. 2 and 3, in conjunction with FIG. 1, disposable portion 500 includes a housing 70 and a disposable electrode assembly 21 that are configured to releasably couple to reusable portion 20. Housing 70 includes a pair of housing halves 70a, 70b configured to matingly engage and releasably encompass at least a portion of one of the shaft members, e.g. shaft member 14. As discussed in more detail hereinbelow, disposable portion 500 includes one or more limited-use features to prevent re-use thereof.

Continuing with reference to FIGS. 2 and 3, housing 70 may also serve to house other features of the forceps 10 which are not discussed in detail herein, e.g, knife 85 and knife actuation mechanism 90 configured to effect advancement of the knife 85 through a knife channel 58 (FIG. 2) defined in one or both electrodes 110, 120 to transect sealed tissue. Alternatively, a cutting electrode (not shown) may be provided on one of jaw members 42, 44 for electrically cutting tissue.

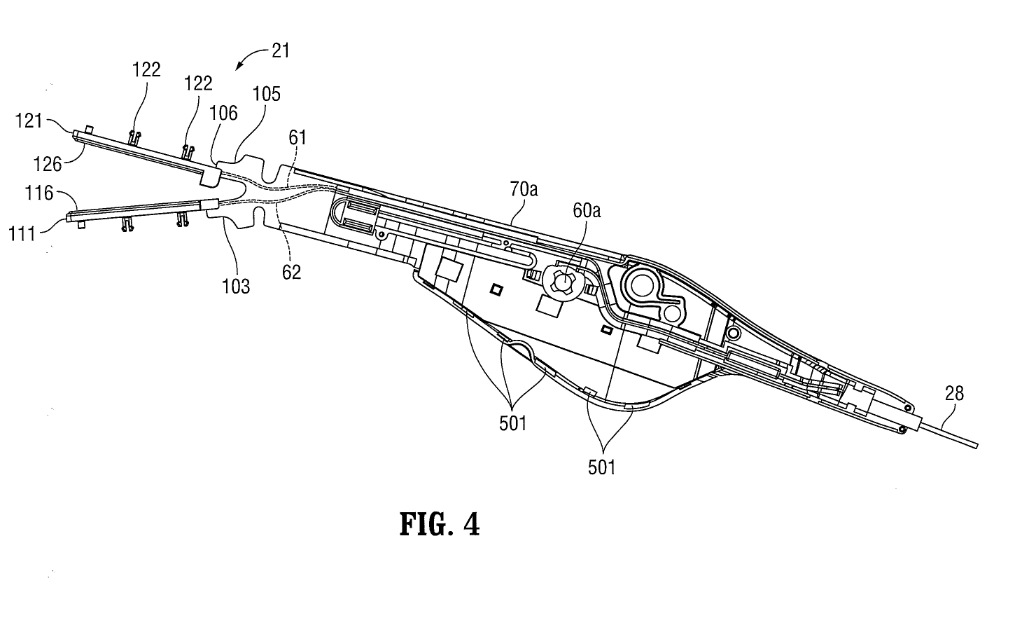

Referring to FIGS. 3-4, an interior of each of housing half 70a, 70b may include a plurality of cooperating mechanical interfaces 501 disposed at various positions to effect mechanical coupling of housing halves 70a, 70b to form housing 70 about shaft 14. It is important to note that housing 70 and the inter-cooperating components therein e.g., knife 85, knife actuator 90, limited use features (as described herein), are all pre-assembled and the entire housing 70 is designed to snap-fit (or otherwise engage) shaft 14. This facilitates assembly of the disposable portion 500 with the reusable portion 20.

It is important to note that in other embodiments, as described in more detail below, the limited-use feature of disposable portion 500 includes frangible mechanical interfaces 501 or similar anchor portions that are configured such that disengagement of housing halves 70a, 70b, e.g., to disengage housing 70 from shaft member 14, requires permanently breaking one or more of the mechanical interfaces 501. As such, upon disassembly, housing halves 70a, 70b are no longer engagable with one another, thereby inhibiting reuse of housing 70. In this instance, more assembly of the disposable portion 500 would be required. Mechanical interfaces 501 may be created using brittle materials such that pulling apart the housing halves 70a, 70b fractures the material forming mechanical interfaces 501. In other embodiments, any suitable structure(s) or mechanism(s) for causing mechanical interfaces 501 to fail upon disassembly, e.g., upon disengaging halves 70a, 70b from one another, may be employed.

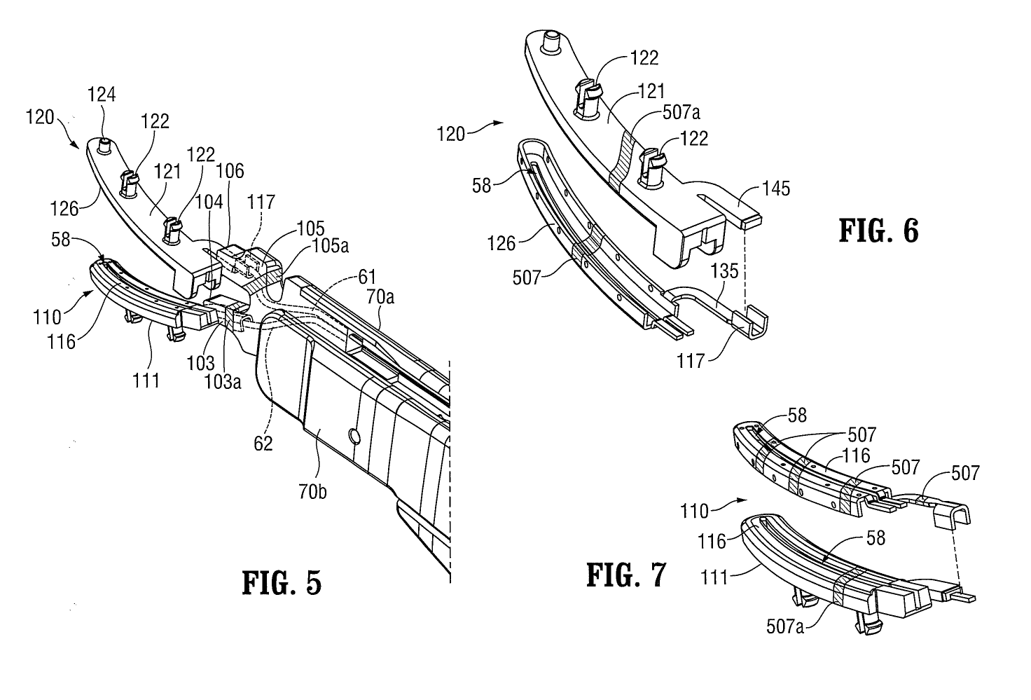

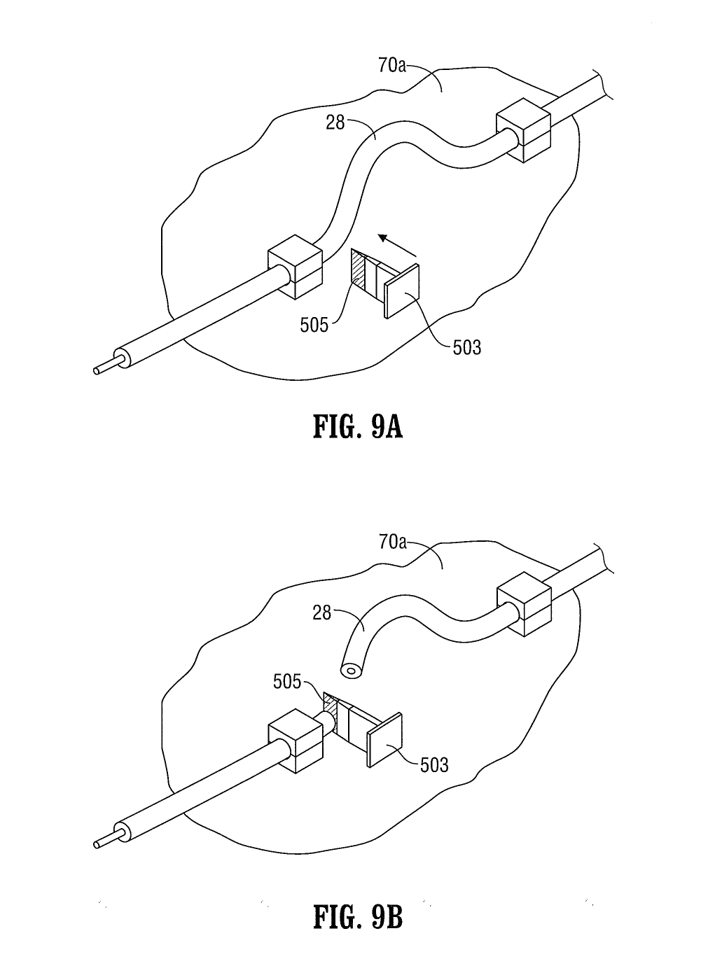

As shown in FIGS. 4-5, a pair of wires 61 and 62 are electrically connected to the electrodes 120 and 110, respectively, and are bundled to form a cable 28 that extends through housing 70 and terminates at a terminal connector 30 (FIG. 1) configured to mechanically and electrically couple to a suitable energy source such as an electrosurgical generator (not shown). The limited-use feature of the disposable portion 500 may include wires 61, 62 and/or cable 28, such that disengaging disposable portion 500 from the forceps 10 causes the wires 61, 62 and/or cable 28 to sever or otherwise become electrically interrupted. For example, with further reference to FIGS. 9A and 9B, a release button 503 provided for disengaging disposable portion 500 from reusable portion 20 may include a blade 505 for severing cable 28 upon disengagement of disposable portion 500 from reusable portion 20. After use, a user would press release button 503 to remove disposable portion 500 from reusable portion 20, and in doing so, would sever cable 28, thereby requiring replacement of disposable portion 500. Mechanical fuses may be used in place of or in conjunction with the wire cutting mechanism shown in FIGS. 9A and 9B, such that the release button 503 breaks a mechanical fuse at a desired location (e.g., at the joint of prong-like portions 103, 105 described below) along the line of electrical conductivity.

Referring now to FIGS. 3-7, electrode assembly 21 includes a generally circular boss member 49 configured to be seated (e.g., friction fit) within a complementary aperture 71 disposed through a distal end of housing half 70a to releasably attach electrode assembly 21 thereto. Electrode assembly 21 is bifurcated such that two prong-like members 103 and 105 extend distally therefrom to support electrodes 110 and 120, respectively. Prong-like members 103, 105 may be flexibly connected at a living hinge or rotatably connected in any other suitable manner.

Prong-like members 103 and 105 may be at least a portion of the limited-use feature of disposable portion 500. For example, as shown in FIG. 5, prong like members 103, 105 may be configured to break apart from the electrode assembly 21 during disengagement of electrode assembly 21 from reusable portion 20. More specifically, prong-like members 103, 105 may be at least partially made of a brittle material, or include a brittle portion 103a, 105a, such that the forces required to disengage electrode assembly 21 from housing 70 are sufficient to fracture the brittle material or portion 103a, 105a. Alternatively, portions 103a, 105a (or the entire prong-like members 103, 105) may be configured to crumple or otherwise deform upon disengagement. Any other suitable structure(s) or mechanism(s) for breaking or deforming prong-like members 103, 105 during disengagement may additionally or alternatively be employed.

Electrode 120 includes an electrically conductive sealing surface 126 configured to conduct electrosurgical energy therethrough and an electrically insulative substrate 121 that serves to electrically insulate jaw member 42 from sealing surface 126. As shown in FIG. 6, and similar to prong-like members 103, 105 described above, the limited-use feature of disposable portion 500 may include at least a portion of the sealing surface 126 and/or substrate 121 such that the sealing surface 126 and/or substrate 121 breaks, is disfigured or is otherwise rendered inoperable upon disengagement from reusable portion 20. Electrode 110 may include any or all of the features of electrode 120. Thus, for purposes of brevity and avoiding repetition, similar features of electrodes 110, 120 will be described only once (with either collective reference to electrodes 110, 120 or the particular components thereof or with reference to only one of electrodes 110, 120 or the particular components thereof).

Substrate 121 of electrode 120 includes a plurality of bifurcated anchor members 122 extending therefrom that are configured to compress during insertion into a corresponding plurality of sockets 43 disposed at least partially through jaw member 42 and subsequently expand to releasably engage corresponding sockets 43 after insertion to couple electrode 120 to jaw member 42. Bifurcated anchor members 122 may be made of any suitable material having a low material failure stress such that removal of the bifurcated anchor members 122 breaks at least a portion of the bifurcated anchor members 122, thus rendering the bifurcated anchor members 122 inoperable for reinsertion into the jaw member 42.

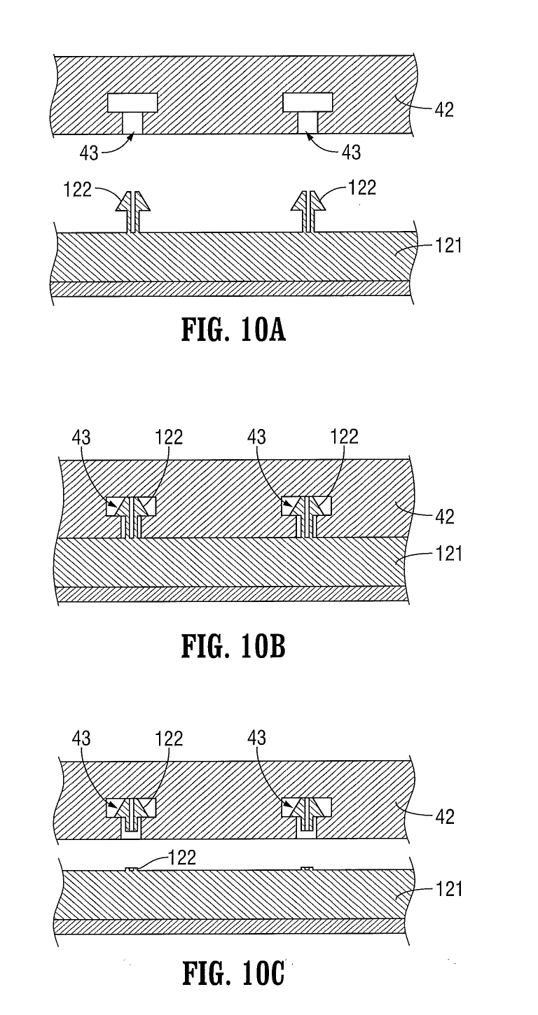

The limited-use feature of the disposable portion 500 may include the anchor members 122. For example, and with additional reference to FIGS. 10A-10C, anchor members 122 are configured to easily engage within sockets 43 (FIGS. 10A and 10B), but are inhibited from being disengaged from sockets 43 unless anchor members 122 are permanently snapped, broken, or otherwise disfigured (FIG. 10C). Anchor members 122 may be made of any suitable material and/or configuration such that a desired removal force may fracture or stretch bifurcated anchors 122 allowing substrate 121 to be disengaged from jaw member 42, while rendering the anchors 122 thereafter unsuitable for engaging the substrate 121 about jaw member 42. In other words, the anchor members 122 may be made of any suitable material and/or may be configured in any suitable fashion that allows anchors 122 to maintain engagement of the substrate 121 and the jaw member 42 during normal use, but upon disengagement the anchors 122 (and, thus, further use of substrate 121) are rendered inoperable.

Referring again to FIGS. 3-7, conductive sealing surface 126 of electrode 120 includes a main body portion and an extension 135 having a wire crimp 117 configured to be inserted into the distal end 106 of prong 105 of electrode assembly 21 and electrically connect to wire 61 disposed therein (FIG. 5). Sealing surface 116 includes similar features as sealing surface 126. Sealing surfaces 116, 126 may be at least a part of the limited-use feature of disposable portion 500. For example, as shown in FIGS. 6 and 7, sealing surface 116, 126 may include at least one crumble zone 507 comprised of a brittle material or a suitable material that allows for the respective sealing surface 116, 126 to crumble upon removal from jaw members 42, 44. In other embodiments, conductive sealing surfaces 116, 126 may separate from substrates 111, 121 when removed from jaw members 42, 44 and may be configured such that sealing surfaces 116, 126 can no longer be engaged to respective substrates 111, 121. Alternatively or additionally, substrates 111,121 may include crumple zones 507a for similar purposes.

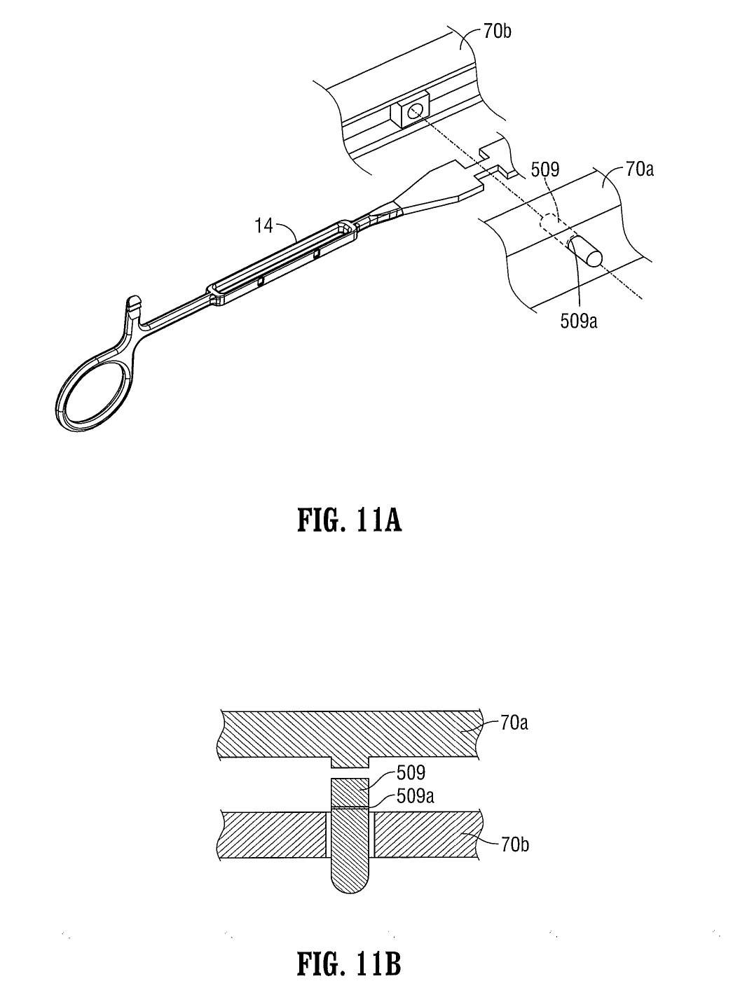

Referring to FIGS. 11A-11B, a release pin 509 may be employed facilitate disengagement of housing 70 from shaft 14. Upon depression of release pin 509, housing 70 is urged apart from shaft 14 to disengage the mechanical snap or latch components retaining housing 70 to shaft 14. Upon depression of release pin 509, the release pin 509 is retained in the depressed position, e.g., via an O-ring 509a surrounding pin 509 and inhibiting return of pin 509, such that housing 70 cannot be reengaged to shaft 14, thereby preventing reuse of disposable portion 500.

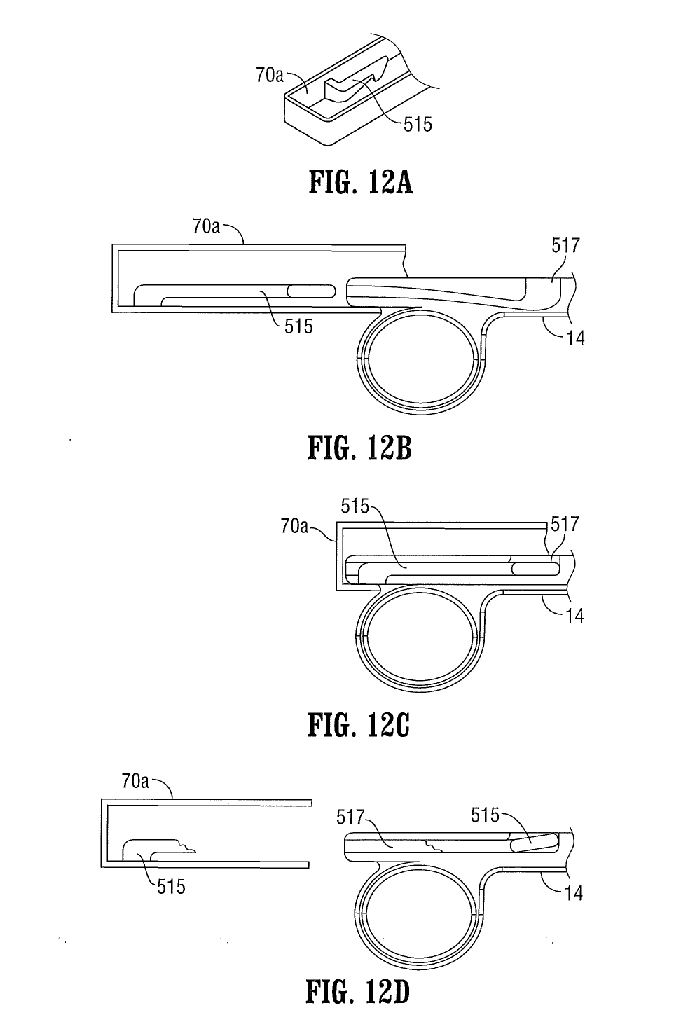

As shown in FIGS. 12A-12D, housing 70 includes a flexible clip 515 configured to anchor housing 70 and, thus, electrode assembly 21, to the reusable portion 20. Further, at least one shaft member 12, 14 (FIG. 1), e.g., shaft member 14, includes a channel 517 for accepting flexible clip 515 to allow housing 70 to snap-fit into engagement about shaft member 14 of reusable portion 20. After insertion (FIG. 12C), the flexible clip 515 is prevented from being removed due to its shape and location in channel 517. The only way to remove the flexible clip 515 (and thus housing 70) from the reusable portion 20 is to break flexible clip 515 by applying a suitable force to fracture flexible clip 515 (FIG. 12D). Flexible clip 515 may be made of any suitable brittle, crumplable, stretchable, or deformable material that allows disengagement under a desired force, but permanently disables flexible clip 515 from reuse.

Referring again to FIGS. 1-3, to electrically control the end effector 24, the housing 70 supports a pair of depressible activation buttons 50a, 50b that are operable by the user to actuate corresponding switches 60a, 60b, (see FIG. 3) respectively, disposed within housing 70. Switches 60a, 60b are electrically interconnected with wires 61, 62, respectively, and serve to initiate and terminate the delivery of electrosurgical energy from a suitable energy source to the end effector 24 to effect tissue treatment. Similar to the embodiments shown in FIGS. 9A-9B, wires 61, 62 may be cut or linked to a mechanical fuse that is destroyed upon disengagement of disposable portion 500 from reusable portion 20, thereby preventing further use of activation buttons 50a, 50b.

As described above, disposable portion 500 is configured for use with an electrosurgical forceps 10, however, the principles disclosed herein regarding limited-use features, designs, and functionalities thereof may be applied to any other desired removable component of a medical device.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawings are presented only to demonstrate certain examples of the disclosure. Other elements, steps, methods and techniques that are insubstantially different from those described above and/or in the appended claims are also intended to be within the scope of the disclosure.

What is claimed is:

1. A forceps, comprising:
a reusable portion; and
a disposable portion configured to operably engage the reusable portion, the disposable portion including at least one limited-use feature, wherein upon disengagement of the disposable portion from the reusable portion, the at least one limited-use feature is transitioned from a first state, permitting engagement of the disposable portion with the reusable portion, to a second state, wherein the limited-use feature inhibits reengagement of the disposable portion with the reusable portion.

2. The forceps of claim 1, wherein the disposable portion is a disposable electrode assembly.

3. The forceps of claim 2, wherein the disposable electrode assembly is configured to conduct electrosurgical energy to tissue.

4. The forceps of claim 1, wherein the reusable portion is a jaw member disposed at a distal end of the forceps, the disposable portion is a jaw substrate that is operably engagable with the jaw member, and wherein the limited-use feature is a bifurcated anchor member disposed on the jaw substrate, the bifurcated anchor member being disposed in the first state before insertion into the jaw member, maintained in the first state after insertion into the jaw member, and transitioned to the second state upon disengagement from the jaw member.

5. The forceps of claim 4, wherein the bifurcated anchor member is made from a material having a low material failure such that removal of the bifurcated anchor member breaks at least a portion of the bifurcated anchor member, thus rendering the bifurcated anchor member inoperable for reinsertion into the jaw member.

6. The forceps of claim 1, wherein the reusable portion is a shaft member of the forceps and the disposable portion is a housing, and wherein the limited-use feature is a frangible anchor member that facilitates attachment of the housing to the shaft member.

7. The forceps of claim 1, wherein transition of the limited-use feature from the first state to the second state renders the disposable portion electrically inoperable.

8. The forceps of claim 1, wherein the disposable portion is configured to be disengaged from the reusable portion via a release member, wherein actuation of the release member transitions the limited-use feature from the first state to the second state.

9. The forceps of claim 1, wherein the limited-use feature further includes at least one mechanical fuse, and wherein, in the second state, the mechanical fuse prevents electrosurgical energy from being applied to tissue.

10. The forceps of claim 9, wherein the at least one mechanical fuse is configured to disconnect or break upon removal of the disposable portion from the reusable portion.

11. A method for preventing reuse of a surgical instrument, comprising the steps of:
providing a forceps comprising a reusable portion and a disposable portion that includes at least one limited-use feature, wherein the disposable portion is engaged with the reusable portion; and
disengaging the disposable portion from the reusable portion, thereby transitioning the limited-use feature from a first state to a second state, wherein the first state permits engagement of the disposable portion with the reusable portion and the second state inhibits reengagement of the disposable portion with the reusable portion.

12. A forceps, comprising:
a reusable portion; and
a disposable portion configured to operably engage the reusable portion, the disposable portion including at least one limited-use feature, wherein upon disengagement of the disposable portion from the reusable portion, the at least one limited-use feature is transitioned from a first state, permitting engagement of the disposable portion with the reusable portion, to a second state, wherein the limited-use feature inhibits reengagement of the disposable portion with the reusable portion, wherein the limited-use feature includes a flexible clip configured to anchor the disposable portion to the reusable portion.

13. The forceps of claim 12, wherein the reusable portion includes a channel defined therein for accepting the flexible clip to allow the disposable portion and the reusable portion to snap-fit into engagement.

14. The forceps of claim 13, wherein the flexible clip is prevented from being removed from the channel without fracturing the flexible clip by applying a suitable force to fracture the flexible clip.

15. The forceps of claim 12, wherein the flexible clip is made of a brittle, crumplable, stretchable, or deformable material that allows disengagement under a desired force, but permanently disables flexible clip from reuse.
